(19) United States
(12) Patent Application Publication
(10) Pub. No.: US 2021/0106646 A1
(43) Pub. Date: Apr. 15, 2021

(54) Combination Of A Novel Topical Gel And Oral Supplements For Healing Diabetic Foot And Other Wounds

(71) Applicant: Smart Products & Services Inc. (dba Aloevive), Bridgewater, NJ (US)

(72) Inventors: Ramendra N. Pandey, Bridgewater, NJ (US); Anand Swaroop, Belle Mead, NJ (US); Usha Pandey, Bridgewater, NJ (US); Ritu R. Pandey, Bridgewater, NJ (US); Richa R. Rane, Edison, NJ (US); John D. Wilson, Powhatan, VA (US)

(73) Assignee: Smart Products & Services Inc. (dba Aloevive), Bridgewater, NJ (US)

(21) Appl. No.: 16/598,072

(22) Filed: Oct. 10, 2019

(51) Int. Cl. A61K 36/886; A61K 31/198; A61K 36/61; A61K 31/16; A61K 33/30; A61K 33/08; A61K 31/34; A61K 31/4166; A61K 31/194; A61K 31/355; A61K 31/4406; A61K 31/728; A61K 47/10; A61K 47/38; A61K 47/46; A61K 9/06

(52) U.S. Cl. CPC A61K 36/886; A61K 9/06; A61K 36/61; A61K 31/16; A61K 33/30; A61K 33/08; A61K 31/34; A61K 31/4166; A61K 31/194; A61K 31/355; A61K 31/4406; A61K 31/728; A61K 47/10; A61K 47/38; A61K 47/46; A61K 31/198

(57) ABSTRACT

Disclosed are topical wound healing compositions useful in the holistic treatment of a patient's wound, particularly diabetic wounds of the foot. Methods of using these formulations topically and in coordinated use with certain oral supplements to improve treatment is also disclosed. In particular, formulations may include one or more of: water, Aloe Barbadensis leaf juice, micro silver, L-Arginine, locust bean gum, and Manuka honey. The topical formulations may be in the form of a gel.

### BACKGROUND OF INVENTION

The invention relates to novel topical wound healing compositions, in some embodiments, augmented with oral supplements, selected and/or formulated to promote various wound healing pathways, and methods of treating wounds, particularly diabetic wounds, especially wounds of the foot.

Chronic diabetic wounds, particularly those of the foot, often do not heal easily. At present, diabetic foot ulcer therapy continues to be an unmet medical need. Since 1997 no significant progress has been made in the field of topical diabetic wound healing gels. The only biopharmaceutical addressing this problem was approved by the US FDA on Dec. 17, 1997¹. The clinical efficacy of this drug was 50% and the placebo was 33%², an increase of 52% in comparison to placebo. Even though this is not a very impressive improvement, for a chronic diabetic foot ulcer patient who is facing the risk of losing a limb, this modest improvement is still a tremendous gift: it increases the probability of retaining the limb. Since 1997 several mono-therapies using biopharmeceuticals³⁻⁵ have been evaluated but none of these have received approval from the US FDA for the treatment of diabetic foot ulcer.

Diabetic foot ulcer patients suffer from a variety of complications such as poor blood flow, bacterial infections and necrosis. Managing all of these issues with a single drug (monotherapy) is difficult. To make matters more complex, many wound healing gels are formulated with synthetic raw materials and active ingredients that manage a few wound-healing deficiencies but fail to successfully address the complete wound healing cascade. And many of the current topical wound healing gel formulations contain synthetic chemical preservatives that can actually slow wound healing⁶, cause allergic reaction⁷, or even cancer⁸. Despite the fact that they can be harmful, synthetic preservatives are still used in the cosmetic formulations because they help to prevent bacterial growth in the product and the product remains useful for a reasonable period.

There are at least two commercially available formulations having a similar stated objective.

### Silvadene

Source: (https://www.webmd.com/drugs/2/drug-4910/silvadene-topical/details)
While this topical agent based on antibiotics is said to be useful, its website acknowledges possible side effects including pain, burning, or itching of the treated skin. Treated skin and mucous membrane (such as the gums) may also become blue/gray in color.

### Regranex

Source: (https://www.rxlist.com/regranex-side-effects-drug-center.htm)
Regranex (becaplermin) Gel is a recombinant human platelet-derived growth factor used to treat lower extremity diabetic ulcers but its website indicates side effects include:

Red skin rash

Burning at the application site.

Rare but serious side effects of Regranex Gel may include allergic reactions such as rash, itching, swelling (especially of the face/tongue/throat), dizziness, and trouble breathing. Tell your doctor if you have severe itching, redness, blistering, peeling, or other skin irritation after using Regranex Gel.

Silvadene contains sulpha drug like moiety or chemical residues. Regranex is preserved by synthetic preservatives like methyl paraben, propyl paraben, and meta cresol.

### SUMMARY OF THE INVENTION

To address these problems, a holistic approach to wound healing has been developed in which a proprietary topical formulation comprises compounds now found to be wound healing agents in unique combinations and in unique amounts, with or without oral supplements, which is used to heal a wide variety of wounds including, but not limited to partial and full thickness diabetic foot wounds. In certain embodiments, wound healing formulations comprises one or more of the following ingredients: aloe barbadensis leaf juice⁶, hyaluronic acid⁷⁻⁸, allantoin¹², niacinamide (vitamin B3)¹³, provitamin B5¹⁴, L-arginine¹⁵⁻¹⁹, zinc oxide²⁰⁻²³, magnesium oxide²⁴, beta glucan²⁵⁻²⁶, silver (average particle size of between about 0.10 and about 100 μm (“micro silver”))^{27, 28} and honey, in particular, Manuka honey²⁹⁻³¹. These formulations develop unique wound healing gel embodiments using novel combinations ingredients having proven wound healing properties⁹⁻³¹. These ingredients are combined to prepare a topical hydrogel that will positively affect various wound healing pathways when applied in a sufficient amount, and in sufficient frequency, to minimizing wound inflammation and promoting rapid wound healing.

In one embodiment a topical formulation is provided comprising water 70% to about 90%), L-arginine (1% to about 20%), guar gum (1% to about 5%) and micro silver (0.1% to about 5%). In another particular embodiment the topical formulation further comprises manuka honey (1.0 to 50.0%).

In another embodiment the topical formulation includes aloe barbadensis leaf juice (10% to about 90%), L-arginine (1% to about 20%), a gelling material such as guar gum (1% to about 5%) and micro silver (0.1% to about 5%). In another particular embodiment the topical formulation further comprises manuka honey (1.0-50.0%).

In another embodiment a topical formulation is provided comprising aloe barbadensis leaf juice (10% to about 90% by weight), allanton (0.01% to about 1%), L-arginine (1% to about 20%), gelling material (Guar gum or any other gelling material, 1-5%), and micro silver (0.1% to about 5%). In another particular embodiment the topical formulation further comprises locust bean gum (1.0-5.0%) In still another embodiment the topical formulation includes aloe barbadensis leaf juice, allanton, L-arginine, micro silver, in the amounts noted above and Manuka honey (1.0-50.0%). And in still another embodiment, these formulations include both locust bean gum (0.5-5.0%) and Manuka honey (1.0-50%).

In a further embodiment, the aloe barbadensis leaf juice is present in an amount of about 50% to about 80% by weight, the locust bean gum in an amount of about 1% to about 5%, the allanton in an amount of about 0.1% to about 0.5%, micro silver is present in an amount of about 0.2% to about 1%, the Manuka honey is present in an amount of about 5% to about 20% by weight and the L-arginine is present in an amount of about 5% to about 10% by weight. (See Table 1.) The topical wound healing formulations of any of these embodiments may be formulated as a cream, ointment, paste, milk, serum, however, it is preferably provided as a gel. In addition, the formulation can be provided as a dry powder that may be poured or sprinkled into a wound to be reconstituted and/or gelled by the liquids in the wound.

In another aspect of the invention the formulations described immediately above are paired with an oral supplement containing L-arginine, turmeric and/or a piperine (piperin or its isomer chavicine). If the oral supplementation includes L-arginine the amount of arginine ranges from between about 3 to about 9 g per day. If turmeric is provided, it is present in an amount of between about 1 and about 2 g per day. If piperine is used, it is provided in an amount of between about 10 mg to about 20 mg per day. Another aspect of the invention is a kit comprising a wound healing formulation for topical application and an oral supplement as described above and herein.

In particular embodiments, these topical formulations consist essentially of the aforementioned ingredients without appreciable amounts of synthetic preservatives such as, without limitation: methyl paraben, propyl paraben and meta cresol, sulpha drug-like moieties or chemical residues.

The wound healing realized from the use of the topical formulations of the invention can be further enhanced by including L-arginine in the topical formulation, and in some embodiments, by oral supplementation with L-arginine and/or turmeric and/or piperine, and combinations thereof. Fortification of topically applied formulation with L-arginine, and/or concomitant supplementation with oral L-arginine, and/or turmeric with piperine, supplemented in the appropriate amounts may provide a significant breakthrough in chronic wound healing.

Another aspect of the invention is a kit comprising: any of the topical wound healing compositions described herein, including, without limitation, aloe barbadensis leaf juice in an amount of about 50% to about 80%, allantoin in an amount of about 0.1% to about 0.5%, micro silver in an amount of about 0.2% to about 1%, L arginine HCl in an amount of about 5.0% to about 10%, and either locust bean gum in an amount of 1%-5% or Manuka honey of at least UMF 20+ in an amount of about 5% to about 20%, (or both locust bean gum and Manuka honey) and optionally one or more of about 5% of a cellulose, up to 5% of a gum, up to about 10% of solvent, up to about 20.0% of a vitamin, all by weight, and at least one oral daily dose comprising at least one of L-arginine in an amount of about 1-30 g, Turmeric in an amount of about 1-10 g and piperine in an amount of about 10-50 mg. These can be divided into a number of dosage forms for daily dosing. For example, the kit could include 2-4 tablets/capsules each containing between about 1 and about 5 g, more preferably between about 1 and about 3 g of L-arginine per dosage form. Turmeric/piperine could be provided in amounts of about 1-5 g/5-25 mg and more preferably 1-3 g/5-15 mg per dosage form and could be included in the same dosage forms that contain the L-arginine or could be provided in 2-4 separate tablets or capsules.

A further aspect of the invention is a method of treating a wound comprising the steps of applying to a wound of a patient a topical wound healing composition as described herein having the ingredients described and exemplified in Table 1. These may include application of formulations comprising, for a nonlimiting example, aloe barbadensis leaf juice in an amount of about 50% to about 90%, allantoin in an amount of about 0.1% to about 0.5%, micro silver in an amount of about 0.2% to about 1%, L-arginine HCl in an amount of about 5.0% to about 10%, at least one of locust bean gum in an amount of about 1-5% or Manuka honey of least UMF 15+ in an amount of about 5% to about 20% (or both locust bean gum and Manuka honey) along with up to about 10% of a cellulose, up to 5% of a gum, up to about 10% of solvent, up to about 20.0% of a vitamin, all by weight, and orally administering to the patient a dosage form including at least one of L-arginine in an amount of about 1-5 g, Turmeric in an amount of about 1-5 g and/or piperine in an amount of about 10-50 mg per dose. The topical wound healing composition is administered topically once or twice a day and the oral dosage form is administered up to 4 times a day.

### DETAILED DESCRIPTION

A list of wound healing ingredients is provided in the tables below. The present invention is not limited to the compositions described in these tables, but encompasses any and all the embodiments within the scope of this disclosure. Table 1 includes the primary active ingredients used in the wound healing formulations of the invention. Other active ingredients may be added in addition to one or more of the active ingredients of Table 1. Table 2 includes additional ingredients or excipients, which, in this instance, are exemplary gel forming materials.

TABLE 1
Wound Healing Active Ingredients

| | General | Specific | More Preferable |
|---|---|---|---|
| | Formulation | Formulations | Formulation |
| Ingredient | % by weight | % by weight | % by weight |
| Aloe Barbadensis | 10.0-90.0 | 20.0-85.0 | 50.0-80.0 |
| Leaf Juice | | | |
| Locust Bean Gum | 0-5.0 | 0.5-5.0 | 1.0-4.0 |
| Allantoin | 0.01-1.0 | 0.05-0.8 | 0.1-0.5 |
| Micro Silver | 0.1-5.0 | 0.2-2.0 | 0.2-1.0 |
| Manuka Honey | 1.0-50.0 | 1.0-25 | 5.0-20.0 |
| Zinc oxide | 0.0-20.0 | 0.1-10.0 | 0.5-5.0 |
| Magnesium | 0.0-20.0 | 0.1-10.0 | 0.5-5.0 |
| Oxide Super low | 0.0-10.0 | 0.1-8.0 | 0.2-5.0 |
| molecular weight | | | |
| hyaluronic acid | | | |
| (SLMW HA) | | | |
| L-Arginine-HCl | 1.0-20.0 | 2.0-15.0 | 5.0-10.0 |
| Hyaluronic Acid | 0.0-3.0 | 0.01-2.0 | 0.2-2.0 |
| Niacinamide | 0.0-20.0 | 1.0-15.0 | 1.0-10.0 |
| (Vitamin B3) | | | |
| Provitamin B5 | 0.0-20.0 | 1.0-15.0 | 1.0-10.0 |
| Beta Glucan | 0.0-10.0 | 0.1-8.0 | 0.5-5.0 |
| Citric Acid | 0.0-20.0 | 0.01-10.0 | 0.2-5.0 |

One formulation of the present invention includes at least some amount of aloe barbadensis leaf juice, allantoin, Manuka honey, L-arginine-HCl and Micro Silver, without synthetic preservatives. These active ingredients are not believed to have been used together and it was not appreciated that this particular combination provides benefits in terms of wound healing that could not have been expected. It has been discovered that by combining these specific ingredients the wound healing potential has been significantly enhanced.

Because of the physical and chemical limitations of these ingredients, it can be difficult to combine these ingredients particularly to produce a wound healing hydrogel or other formulation like an occlusive ointment, and the art did not recognize the potential synergy of these ingredients when used for a topical wound healing formulation. A formulation strategy has therefore been developed that combines these wound healing agents in a topical formulation, and in particular, a hydrogel formulation. The resultant topical formulations provide rapid wound hydration, anti-microbial activity, and anti-inflammatory effect to the wound. Furthermore, the ingredients present in the formulation take advantage of the wound milieu present at the wound site. A freshly debrided wound has high concentration of iNOS. The L-arginine present in the formulation and the iNOS present in the freshly debrided wound react and produce non specific signal transduction agent nitric oxide (NO). NO being a non specific signal transduction agent, sends signals to brain and elicits increased blood flow to the wound site. Increased blood flow at the wound site is likely to increase the platelets and neutrophils and in turn, platelet derived growth factor secretion is likely to be enhanced at the wound site and this has potential to enhance wound healing. It is important to mention at this point that after wound debridement, as time passes, iNOS level at the wound site decreases and arginase level increases. The same wound healing agent L-arginine, present in the formulation, gets metabolized differently with the enzyme arginase. Arginase metabolizes L-arginine into urea and ornithine. Ornithine further gets metabolized into proline which is essential for collagen synthesis which in turn facilitates wound closer.

Aloe barbadensis leaf juice⁹ acts as a moisturizer and also as an anti-inflammatory agent. The high molecular weight carbohydrates present in the aloe act as immunomodulator. For better therapeutic outcome, it is essential to use aloe with higher concentration of high molecular weight carbohydrates.

Allantoin¹⁰ is a natural ingredient found in cow urine and some plants. It acts as a potent wound healing agent.

L-arginine¹⁵⁻¹⁹ is a conditionally essential amino acid. In healthy individuals body synthesizes needed L-arginine. However, in case of certain illnesses (demands for arginine increases and arginine supplementation plays a beneficial role. L-arginine plays a major role in wound healing. A freshly debrided wound contains high concentrations of the enzyme inducible N-oxide synthase (iNOS)³² which metabolizes L-arginine to nitric oxide and citruline. Nitric oxide, a non-specific signal transduction agent, signals the brain to send platelets, neutrophils, and macrophages to the wound site to augment the wound healing process. After a few days the N-oxide synthase level decreases in the wound and the level of arginase increases. Arginase also requires L-arginine as a substrate and metabolizes it to urea and ornithine. Ornithine, which is subsequently converted to proline, participates in collagen synthesis, a major process in wound healing.

For a healing wound there is a persistent demand for L-arginine at the wound site³³⁻³⁴. Animal studies have shown decreased arginine levels in the wound fluid³⁵⁻³⁶ suggestive of a high local demand for arginine. Oral arginine taken alone may not be very effective in fulfilling the increased need for arginine at the wound site. Therefore, a topical formulation containing L-arginine, which will meet the local demand for increased L-arginine at the wound site, is highly desirable. As a consequence of the increased consumption of L-arginine by N-oxide synthase and arginase, the local L-arginine level at the wound site may decrease over time. This decrease may be effectively managed by reapplying the topical formulation according to an appropriate time schedule. But it is believed that using a formulation of the invention supplemented with oral L-arginine, and in particular, the use of oral supplementation with L-arginine with the use of such formulation that also includes L-arginine, can maximize wound healing.

Manuka Honey²⁹⁻³¹ is a special honey variety which is produced by the bees in the Eastern New Zealand region feeding on Manuka bushes. Apart from traditional hydrogen peroxide present in all honeys, this honey also contains methylglyoxal (MGO) which imparts special antibacterial properties to this honey. It is very important to use only the Manuka honey which is certified to be Unique Manuka Factor Honey Association (“UMFHA”) as having a Unique Manuka Factor (“UMF”) of 16+, preferably 20+ or higher. In the formulation, Manuka honey provides antibacterial and/or anti-inflammatory properties. It is believed that the compound responsible for the UMF score is methylglyoxal.

In any of the foregoing formulations and methods, micro silver acts both as a preservative and, upon application to the wound site, acts as a broad-spectrum anti-microbial²⁸. The anti-microbial power of micro silver comes from its ability to generate Ag⁺ ions, under aqueous conditions, from pure silver (Ag⁰). Ag⁺ kills microbes via three different mechanisms. It inhibits cellular ion transport by blocking the ion channels, inactivates intracellular enzymes by binding to them or intercalates with DNA and stops DNA replication. All these three mechanisms eventually lead to cell death. The present invention relates to a wound healing composition with significant antibacterial and anti-inflammatory actions, and including the benefit of promoting enhanced collagen synthesis. In some embodiments, natural, multifunctional, highly porous, medical grade, micro silver³⁷ that is applied as part of the formulations applied directly to a wound²⁷⁻²⁸. This helps in minimizing the bacterial load at the wound site, which further promotes wound healing.

Micro silver^{27, 28} is an innovative form of pure metallic silver and it is manufactured using a purely physical, gas phase process, which produces highly pure, elemental silver without any ionic silver contamination. The average particle size of this silver measured by volume can range from about 0.10 to about 100 μm, but is preferably about 10 micrometer. When applied on to the skin, micro silver collects mainly at the pore level and in the skin folds. The silver ions are released on to the surface of the skin and may thus react with the surrounding bacteria and yeast/fungi. Preferably, because of the particle size (average size 10 micrometer) it does not penetrate the skin and is always available on the skin surface to produce silver ions in presence of moisture. Micro silver plays dual role in the formulation it acts as a preservative and onto the skin, in presence of moisture, acts as antimicrobial agent by generating Ag+ and keeps the skin healthy. Other forms of silver and other micro silver products may also be used.

In still other embodiments, the formulation includes the five ingredients noted above, and in addition one or more of the ingredients such as hyaluronic acid^{7, 8}, super low molecular weight (SLMW) hyaluronic acid, oxides (zinc and magnesium)²⁰⁻²⁴. Indeed, formulations useful in accordance with the invention comprise: aloe barbadensis leaf juice, allantoin, micro silver⁵, Manuka Honey, L-arginine-HCL, hyaluronic acid, SLMW hyaluronic acid, and one or more gelling agents such as, without limitation, carboxymethyl cellulose, guar gum, xanthan gum, bentonite clay without synthetic preservatives. Various attributes of above ingredients are provided below:

Hyaluronic Acid¹⁰ is a high molecular weight polysaccharide and it is a very potent moisturizer. Because of its high molecular weight it does not penetrate the skin

Hyaluronic Acid SLMW is a hydrolyzed form of Hyaluronic acid and its molecular weight range is 8-15 killo dalton. Hyaluronic acid (sodium hyaluronate) is a natural polysaccharide (sugar) that occurs in body tissues including the skin providing hydration, stability, and lubrication. The SLMW (super low-molecular-weight) form is made by enzymatically cleaving high-molecular weight hyaluronic acid into small fragments (8-15 kDa). It is a off-white, water-soluble, odorless, fluffy powder. Other forms of hyaluronic acid may also be used.

A number of other active ingredients that may be present are identified in Table 1. These include beta glucan^{25, 26} which is a high molecular weight carbohydrate and it promotes wound healing. It increases macrophage infiltration, which stimulates tissue granulation, collagen deposition and re-epithelialization. Its preferred source is from the yeast.

One or more of magnesium and zinc oxides²⁰⁻²⁴ or salts may be used in the formulations to provide enhanced enzymatic activity to support wound healing. The average particle size for these two elements should be in micro meter range.

These formulations can also include other ingredients such as vitamins, pro-vitamins, glucans such as beta-glucan, stabilizers and anti-oxidants such as citric and ascorbic acids or tocopherol.

Excipients including fillers, viscosity modifiers, emollients, humectants pH adjusting substances, buffers, carriers and solvents such as glycerol and water may also be present. Some exemplary excipients include those found in Table 2. Particularly preferred carriers are gel-formers and materials useful in producing a gel formulation. These can include, for example, celluloses like carboxymethyl cellulose, glycerol, water and gums. Gelling and thickening agents like include gums like xanthan gum, guar gum, locust bean gum and bentonite clay. The amount of clay used can be up to 5%. The amount of gum used can be up to about 3% depending on how many gums are used. Generally, the amount of gum used over and above any locust bean gum used, can be as high as 3% by weight. If the formulation also includes locust bean gum, the total amount of all gums may be up to about 5% by weight.

These are used in varying amounts depending upon a number of factors including the desired use of the formulation (location of its use), the desired viscosity, desired lubricity, desired porosity, and the like. They are conventional agents used in conventional amounts which may vary as is determinable in the industry based on the specific properties of the individual materials selected. Exemplary materials and amounts that can be used when producing gel formulations in accordance with the present inventions include those shown in Table 2.

TABLE 2
Exemplary excipients

| | | | More |
|---|---|---|---|
| | | Preferable | Preferable |
| | | Formulation | Formulation |
| | Formulation | % by | % by |
| Ingredient | % by weight | weight | weight |
| Carboxymethyl | 0.0-5.0 | 0.1-5.0 | 0.5.0-4.0 |
| Cellulose | | | |
| Xanthan gum | 0-5.0 | 0.5-5.0 | 1.0-4.0 |
| Guar gum | 0-5.0 | 0.5-5.0 | 1.0-4.0 |
| Bentonite clay | 0.0-5.0 | 0.1-4.0 | 0.5-3.0 |
| Glycerol | 0.0-10.0 | 1.0-8.0 | 3.0-5.0 |
| Water | q.s. | q.s. | q.s. |

The formulations of the present invention can be adjusted in terms of their properties depending on how and where they are to be applied. The formulations could be relatively viscous so that it can be smeared into a wound, or can be relatively more solid to form a conforming pad to be placed over a wound. But they are all to be placed in contact with the skin and/or an open wound so they must be of a suitable pH, they must be made using medical or pharmaceutical grade ingredients, and they must not needlessly increase the discomfort of the patient. A preferred pH for the described formulation is between 4.8 to 7.4. If not provided as a topical gel, known formulations for preparing ointments, creams, serums, salves, pastes, and the like, may be used in conventional amounts as long as they are capable of delivering the active ingredients discussed herein.

Examples 1-12 propose particular formulations that can be made pursuant to various aspects of the invention. However, it will be appreciated that the amounts of the ingredients, and particularly the active ingredients, recited in these examples can be varied considerably as shown in Tables 3-14 below.

TABLE 3

| | | Preferable | More Preferable | Formulation |
|---|---|---|---|---|
| | | Formulation | Formulation | of Example |
| | Formulation | % by | % by | 1, % |
| Ingredient | % by weight | weight | weight | by weight |
| Water | 10.0-95.0 | 20.0-90.0 | 60.0-90.0 | 85.3 |
| Micro Silver | 0.1-5.0 | 0.2-2.0 | 0.2-1.0 | 0.2 |
| Guar gum | 0-5.0 | 0.5-5.0 | 1.0-4.0 | 2.5 |
| L-Arginine- | 1.0-20.0 | 2.0-15.0 | 5.0-10.0 | 6.0 |
| HCl | | | | |
| Glycerol | 1.0-10.0 | 2.0-8.0 | 3.0-6.0 | 5.0 |
| Citric Acid/ | As needed for | As needed for | As needed for | As needed |
| NaOH | adjusting | adjusting | adjusting | for adjusting |
| | pH 4.8-7.4 | pH 4.8-7.4 | pH 4.8-7.4 | pH 4.8-7.4 |

TABLE 4

| | | Preferable | More | Formulation |
|---|---|---|---|---|
| | | Formulation | Preferable | of Example |
| | Formulation | % by | Formulation | 2, % |
| Ingredient | % by weight | weight | % by weight | by weight |
| Water | 10.0-95.0 | 20.0-90.0 | 60.0-90.0 | 75.3 |
| Micro Silver | 0.1-5.0 | 0.2-2.0 | 0.2-1.0 | 0.2 |
| Guar gum | 0-5.0 | 0.5-5.0 | 1.0-4.0 | 2.5 |
| L-Arginine- | 1.0-20.0 | 2.0-15.0 | 5.0-10.0 | 6.0 |
| HCl | | | | |
| Glycerol | 1.0-10.0 | 2.0-8.0 | 3.0-6.0 | 5.0 |
| Manuka | 1.0-50.0 | 1.0-25 | 5.0-20.0 | 10 |
| Honey | | | | |
| Citric | As needed | As needed | As needed | As needed |
| Acid/ | for adjusting | for adjusting | for adjusting | for adjusting |
| NaOH | pH 4.8-7.4 | pH 4.8-7.4 | pH 4.8-7.4 | pH 4.8-7.4 |

TABLE 5

| | | Preferable | More | Formulation |
|---|---|---|---|---|
| | | Formulation | Preferable | of Example |
| | Formulation | % by | Formulation | 3, % |
| Ingredient | % by weight | weight | % by weight | by weight |
| Aloe | 10.0-95.0 | 20.0-90.0 | 60.0-90.0 | 85.3 |
| Barbadensis | | | | |
| Leaf Juice | | | | |
| Micro Silver | 0.1-5.0 | 0.2-2.0 | 0.2-1.0 | 0.2 |
| Guar gum | 0-5.0 | 0.5-5.0 | 1.0-4.0 | 2.5 |
| L-Arginine- | 1.0-20.0 | 2.0-15.0 | 5.0-10.0 | 6.0 |
| HCl | | | | |
| Glycerol | 1.0-10.0 | 2.0-8.0 | 3.0-6.0 | 5.0 |
| Citric Acid/ | As needed | As needed | As needed | As needed |
| NaOH | for adjusting | for adjusting | for adjusting | for adjusting |
| | pH 4.8-7.4 | pH 4.8-7.4 | pH 4.8-7.4 | pH 4.8-7.4 |

TABLE 6

| | | Preferable | More | Formulation |
|---|---|---|---|---|
| | Formulation | Formulation | Preferable | of Example |
| | % by | % by | Formulation | 4, % |
| Ingredient | weight | weight | % by weight | by weight |
| Aloe | 10.0-95.0 | 20.0-90.0 | 60.0-90.0 | 75.3 |
| Barbadensis | | | | |
| Leaf Juice | | | | |
| Micro Silver | 0.1-5.0 | 0.2-2.0 | 0.2-1.0 | 0.2 |
| Guar gum | 0-5.0 | 0.5-5.0 | 1.0-4.0 | 2.5 |
| L-Arginine- | 1.0-20.0 | 2.0-15.0 | 5.0-10.0 | 6.0 |
| HCl | | | | |
| Glycerol | 1.0-10.0 | 2.0-8.0 | 3.0-6.0 | 5.0 |
| Manuka Honey | 1.0-50.0 | 1.0-25 | 5.0-20.0 | 10 |
| Citric Acid/ | As needed | As needed | As needed | As needed |
| NaOH | for adjusting | for adjusting | for adjusting | for adjusting |
| | pH 4.8-7.4 | pH 4.8-7.4 | pH 4.8-7.4 | pH 4.8-7.4 |

TABLE 7

| | | Preferable | More Preferable | Formulation |
|---|---|---|---|---|
| | | Formulation | Formulation | of Example |
| | Formulation | % by | % by | 5, % |
| Ingredient | % by weight | weight | weight | by weight |
| Aloe | 10.0-90.0 | 20.0-85.0 | 60.0-80.0 | 82.9 |
| Barbadensis | | | | |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Locust | 0-5.0 | 0.5-5.0 | 1.0-4.0 | 2.5 |
| bean gum | | | | |
| Zinc oxide | 0.0-20.0 | 0.1-10.0 | 0.2-1.0 | 0.2 |
| Magnesium | 0.0-20.0 | 0.1-10.0 | 0.2-1.0 | 0.2 |
| Oxide | | | | |
| Super low | 0.0-10.0 | 0.1-8.0 | 0.1-1.0 | 0.2 |
| molecular | | | | |
| weight | | | | |
| hyaluronic | | | | |
| acid (SLMW | | | | |
| HA) | | | | |
| L-Arginine- | 1.0-20.0 | 2.0-15.0 | 5.0-10.0 | 6.0 |
| HCl | | | | |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 5.0 |
| Citric Acid/ | As needed | As needed | As needed | As needed |
| NaOH | for | for | for | for |
| | adjusting | adjusting | adjusting | adjusting |
| | pH 4.8-7.4 | pH 4.8-7.4 | pH 4.8-7.4 | pH 4.8-7.4 |

TABLE 8

| | | Preferable | More | Formulation |
|---|---|---|---|---|
| | | Formulation | Preferable | of Example |
| | Formulation | % by | Formulation | 6, % |
| Ingredient | % by weight | weight | % by weight | by weight |
| Aloe Barbadensis | 10.0-90.0 | 20.0-85.0 | 60.0-80.0 | 67.9 |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Locust Bean gum | 0-5.0 | 0.5-5.0 | 1.0-4.0 | 2.5 |
| Manuka Honey | 1.0-50.0 | 1.0-25 | 5.0-20.0 | 15 |
| Zinc oxide | 0.0-20.0 | 0.1-10.0 | 0.2-1.0 | 0.2 |
| Magnesium | 0.0-20.0 | 0.1-10.0 | 0.2-1.0 | 0.2 |
| Oxide | | | | |
| Super low | 0.0-10.0 | 0.1-8.0 | 0.1-1.0 | 0.2 |
| molecular | | | | |
| weight hyaluronic | | | | |
| acid (SLMW HA) | | | | |
| L-Arginine-HCl | 1.0-20.0 | 2.0-15.0 | 5.0-10.0 | 6.0 |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 5.0 |
| Citric Acid/ | As needed | As needed | As needed | As needed |
| NaOH | for | for | for | for |
| | adjusting | adjusting | adjusting | adjusting |
| | pH 4.8-7.4 | pH 4.8-7.4 | pH 4.8-7.4 | pH 4.8-7.4 |

TABLE 9

| | | Preferable | More | Formulation |
|---|---|---|---|---|
| | | Formulation | Preferable | of Example |
| | Formulation | % by | Formulation | 7, % |
| Ingredient | % by weight | weight | % by weight | by weight |
| Aloe Barbadensis | 10.0-90.0 | 20.0-85.0 | 60.0-80.0 | 82.9 |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Xanthan gum | 0-5.0 | 0.5-5.0 | 1.0-4.0 | 2.5 |
| Zinc oxide | 0.0-20.0 | 0.1-10.0 | 0.2-1.0 | 0.2 |
| Magnesium Oxide | 0.0-20.0 | 0.1-10.0 | 0.2-1.0 | 0.2 |
| Super low | 0.0-10.0 | 0.1-8.0 | 0.1-1.0 | 0.2 |
| molecular | | | | |
| weight hyaluronic | | | | |
| acid (SLMW HA) | | | | |
| L-Arginine-HCl | 1.0-20.0 | 2.0-15.0 | 5.0-10.0 | 6.0 |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 5.0 |
| Citric acid/NaOH | As needed | As needed | As needed | As needed |
| | for | for | for | for |
| | adjusting | adjusting | adjusting | adjusting |
| | pH 4.8-7.4 | pH 4.8-7.4 | pH 4.8-7.4 | pH 4.8-7.4 |

TABLE 10

| | | Preferable | More | Formulation |
|---|---|---|---|---|
| | Formulation | Formulation | Preferable | of Example |
| | % by | % by | Formulation | 8, % |
| Ingredient | weight | weight | % by weight | by weight |
| Aloe Barbadensis | 10.0-90.0 | 20.0-85.0 | 60.0-80.0 | 67.9 |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Xanthan gum | 0-5.0 | 0.5-5.0 | 1.0-4.0 | 2.5 |
| Manuka Honey | 0.0-50.0 | 1.0-25 | 5.0-20.0 | 15 |
| Zinc oxide | 0.0-20.0 | 0.1-10.0 | 0.2-1.0 | 0.2 |
| Magnesium Oxide | 0.0-20.0 | 0.1-10.0 | 0.2-1.0 | 0.2 |
| Super low | 0.0-10.0 | 0.1-8.0 | 0.1-1.0 | 0.2 |
| molecular | | | | |
| weight hyaluronic | | | | |
| acid (SLMW HA) | | | | |
| L-Arginine-HCl | 1.0-20.0 | 2.0-15.0 | 5.0-10.0 | 6.0 |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 5.0 |
| Citric acid/ | As needed | As needed | As needed | As needed |
| NAOH | for | for | for | for |
| | adjusting | adjusting | adjusting | adjusting |
| | pH 4.8-7.4 | pH 4.8-7.4 | pH 4.8-7.4 | pH 4.8-7.4 |

TABLE 11

| | | Preferable | More | Formulation |
|---|---|---|---|---|
| | | Formulation | Preferable | of Example |
| | Formulation | % by | Formulation | 9, % by |
| Ingredient | % by weight | weight | % by weight | weight |
| Aloe Barbadensis | 10.0-90.0 | 20.0-85.0 | 60.0-80.0 | 82.9 |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Guar gum | 0-5.0 | 0.5-5.0 | 1.0-4.0 | 2.5 |
| Zinc oxide | 0.0-20.0 | 0.1-10.0 | 0.2-1.0 | 0.2 |
| Magnesium Oxide | 0.0-20.0 | 0.1-10.0 | 0.2-1.0 | 0.2 |
| Super low | 0.0-10.0 | 0.1-8.0 | 0.1-1.0 | 0.2 |
| molecular | | | | |
| weight hyaluronic | | | | |
| acid (SLMW HA) | | | | |
| L-Arginine-HCl | 1.0-20.0 | 2.0-15.0 | 5.0-10.0 | 6.0 |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 5.0 |
| Citric acid/NaOH | As needed | As needed | As needed | As needed |
| | for | for | for | for |
| | adjusting | adjusting | adjusting | adjusting |
| | pH 4.8-7.4 | pH 4.8-7.4 | pH 4.8-7.4 | pH 4.8-7.4 |

TABLE 12

| | | Preferable | More | Formulation |
|---|---|---|---|---|
| | Formulation | Formulation | Preferable | of Example |
| | % by | % by | Formulation | 10, % by |
| Ingredient | weight | weight | % by weight | weight |
| Aloe | 10.0-90.0 | 20.0-85.0 | 60.0-80.0 | 67.9 |
| Barbadensis | | | | |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Guar gum | 0-5.0 | 0.5-5.0 | 1.0-4.0 | 2.5 |
| Manuka Honey | 0.0-50.0 | 1.0-25 | 5.0-20.0 | 15 |
| Zinc oxide | 0.0-20.0 | 0.1-10.0 | 0.2-1.0 | 0.2 |
| Magnesium | 0.0-20.0 | 0.1-10.0 | 0.2-1.0 | 0.2 |
| Oxide | | | | |
| Super low | 0.0-10.0 | 0.1-8.0 | 0.1-1.0 | 0.2 |
| molecular | | | | |
| weight | | | | |
| hyaluronic | | | | |
| acid (SLMW | | | | |
| HA) | | | | |
| L-Arginine- | 1.0-20.0 | 2.0-15.0 | 5.0-10.0 | 6.0 |
| HCl | | | | |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 1.0 |
| Citric acid/ | As needed | As needed | As needed | As needed |
| NaOH | for adjusting | for adjusting | for adjusting | for adjusting |
| | pH 6.8-7.4 | pH 6.8-7.4 | pH 6.8-7.4 | pH 6.8-7.4 |

TABLE 13

| | | Preferable | More | Formulation |
|---|---|---|---|---|
| | Formulation | Formulation | Preferable | of Example |
| | % by | % by | Formulation | 11, % by |
| Ingredient | weight | weight | % by weight | weight |
| Aloe Barbadensis | 10.0-90.0 | 20.0-85.0 | 60.0-80.0 | 82.9 |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Carboxymethyl | 0-5.0 | 0.5-5.0 | 1.0-4.0 | 2.5 |
| cellulose | | | | |
| Zinc oxide | 0.0-20.0 | 0.1-10.0 | 0.2-1.0 | 0.2 |
| Magnesium Oxide | 0.0-20.0 | 0.1-10.0 | 0.2-1.0 | 0.2 |
| Super low | 0.0-10.0 | 0.1-8.0 | 0.1-1.0 | 0.2 |
| molecular | | | | |
| weight hyaluronic | | | | |
| acid (SLMW HA) | | | | |
| L-Arginine-HCl | 1.0-20.0 | 2.0-15.0 | 5.0-10.0 | 6.0 |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 5.0 |
| Citric acid/NaOH | As needed | As needed | As needed | As needed |
| | for | for | for | for |
| | adjusting | adjusting | adjusting | adjusting |
| | pH 4.8-7.4 | pH 4.8-7.4 | pH 4.8-7.4 | pH 4.8-7.4 |

TABLE 14

| | | Preferable | More | Formulation |
|---|---|---|---|---|
| | Formulation | Formulation | Preferable | of Example |
| | % by | % by | Formulation | 12, % by |
| Ingredient | weight | weight | % by weight | weight |
| Aloe | 10.0-90.0 | 20.0-85.0 | 60.0-80.0 | 67.9 |
| Barbadensis | | | | |
| Leaf Juice | | | | |
| Allantoin | 0.01-1.0 | 0.05-0.8 | 0.1-0.5 | 0.3 |
| Micro Silver | 0.1-5.0 | 0.2-2.0 | 0.2-1.0 | 0.2 |
| Carboxymethyl | 0-5.0 | 0.5-5.0 | 1.0-4.0 | 2.5 |
| cellulose | | | | |
| Manuka Honey | 0.0-50.0 | 1.0-25 | 5.0-20.0 | 15 |
| Zinc oxide | 0.0-20.0 | 0.1-10.0 | 0.2-1.0 | 0.2 |
| Magnesium | 0.0-20.0 | 0.1-10.0 | 0.2-1.0 | 0.2 |
| Oxide | | | | |
| Super low | 0.0-10.0 | 0.1-8.0 | 0.1-1.0 | 0.2 |
| molecular | | | | |
| weight | | | | |
| hyaluronic | | | | |
| acid (SLMW | | | | |
| HA) | | | | |
| L-Arginine- | 1.0-20.0 | 2.0-15.0 | 5.0-10.0 | 6.0 |
| HCl | | | | |
| Niacinamide | 0.5-10.0 | 0.75-8.0 | 1.0-5.0 | 1.0 |
| (Vitamin B3) | | | | |
| Provitamin B5 | 0.5-10.0 | 0.75-8.0 | 1.0-5.0 | 1.0 |
| Beta Glucan | 0.5-10.0 | 0.5-8.0 | 0.5-5.0 | 0.5 |
| Glycerol | 1.0-10.0 | 2.0-8.0 | 3.0-6.0 | 5.0 |
| Citric acid/ | As needed | As needed | As needed | As needed for |
| NaOH | for adjusting | for adjusting | for adjusting | adjusting pH |
| | pH 4.8-7.4 | pH 4.8-7.4 | pH 4.8-7.4 | 4.8-7.4 |

The amount of wound healing formulation used in accordance with the present invention can vary with many factors known to persons of ordinary skill in this art. The composition and concentration of the actives contained in the formulation, whether or not it is being administered along with oral supplementation, the size and nature of the wound all can play a role in how much of the formulation is applied and how frequently and for how long. As one example, however, approximately 0.5 g of the gel formulation is applied per sq cm of the wound area. In one embodiment, the formulation should be applied once a day for the first 7 days followed by alternate days application. The wound should be cleaned with normal saline or mild soap prior to each new application of the formulation.

In one embodiment, wound care and treatment is accomplished using both external formulation application and by providing various important factors to the body system internally as well. Gels, those including one or more of L-arginine, aloe barbadensis and those without any of them, can be applied externally while one or more of L-arginine, turmeric and/or piperine is administered to the subject orally as well. The gel and the oral doses do not have to be given on any particularly coordinated schedule—i.e. eat a certain amount of 1-arginine within 1 hour of application of a gel formulation of the invention. Oral L-arginine should be given up to 30 g per day such as 1-3 g three times a day. Turmeric/piperine should be given up to 10 g/50 mg per day such as 1 capsule (1 gm turmeric+10 mg piperine) three times a day. These ingredients may be given in the form of any oral delivery device such as a tablet, capsule, caplet, powder, spray, film and liquid, such as a syrup. However, preferably they are given as a capsule or an oral solution.

In one embodiment, patients should take 1 capsule of oral L-arginine (1000 mg) and one capsule of turmeric (1000 mg) mixed with peprine (10 mg) three times a day. The patients may also take, instead of oral capsules, equivalent dose of the L-arginine and/or Turmeric with peprine in the form of a solution rather than a capsule.

### Examples

The formulations 1 & 3 are prepared by using either water or aloe barabadensis leaf juice (85.3%) along with micro silver (0.2%), guar gum (2.5%), L-arginine-HCl (6%), Glycerol (6%). The formulations 2 & 4 are prepared by reducing the water or aloe barbadensis leaf juice from the formulations 1 & 3 by 10% and adding 10% of Manuka honey to each and rest of the ingredients remained the same. The formulations of 5, 7, 9 & 11 are prepared by using 4 different gums (5-Locust bean gum, 7-Xanthan gum, 9-Guar gum and 11-Carboxymethyl cellulose) and the formulations of examples 6, 8, 10, & 12 are prepared by reducing the amount of Aloe barbadensis leaf juice from 82.9% to 67.9% and adding Manuka honey at 15%. The nature of the ingredients used in these formulations, should not provoke any allergic reactions unless that particular subject belongs to groups who are predisposed to be allergic to these ingredients. Since these formulations use mainly natural ingredients, they may also be considered safe as ingredients.

Example 1: Water 85.3%; Micro silver—0.2%; Guar gum—2.5%; L-Arginine-HCl—6%; Glycerol—5%; Citric Acid to adjust pH to between 6.0 and 7.4. Procedure for making the formulations: Water or in other examples Aloe barbadensis leaf juice is added in the mixing vessel first. While vigorously stirring the liquid, remaining ingredients are added sequentially. Following the addition of all the ingredients, the resultant gel is mixed for another hour. Upon completion of the mixing, the gel pH was measured and, if needed, adjusted with citric acid/NaOH.

Example 2: Water—75.3; Micro silver—0.2%; Guar gum—2.5%; L-Arginine-HCl—6%; Glycerol—6%; Manuka honey 10%; Citric Acid to adjust pH to between 6.0 and 7.4.

Example 3: Aloe Barbadensis Leaf Juice—85.3%; Micro silver—0.2%; Guar gum—2.5%; L-Arginine-HCl—6%; Glycerol—6%; Citric Acid to adjust pH to between 6.8 and 7.4.

Example 4: Aloe Barbadensis Leaf Juice—75.3%; Micro silver—0.2%; Guar gum—2.5%; L-Arginine-HCl—6%; Glycerol—6%; Citric Acid to adjust pH to between 6.0 and 7.4.

Example 5: Aloe Barbadensis Leaf Juice—82.9%; Allantoin—0.3%; Micro silver—0.2%; Locust bean gum—2.5%; Zinc oxide—0.2%; Magnesium Oxide—0.2%; SLMW HA—0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4.

Example 6: Aloe Barbadensis Leaf Juice—67.9%; Allantoin—0.3%; Micro silver—0.2%; Locust bean gum—2.5%; Manuka honey—15%; Zinc oxide—0.2%; Magnesium Oxide—0.2%; SLMW HA—0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4. This formulation can be made using the process described generally in Example 1.

Example 7: Aloe Barbadensis Leaf Juice—82.9%; Allantoin—0.3%; Micro silver—0.2%; Xanthan gum—2.5%; Zinc oxide—0.2%; Magnesium Oxide—0.2%; SLMW HA—0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4. This formulation can be made using the process described generally in Example 1.

Example 8: Aloe Barbadensis Leaf Juice—67.9%; Allantoin—0.3%; Micro silver—0.2%; Xanthan gum—2.5%; Manuka honey—15%; Zinc oxide—0.2%; Magnesium Oxide—0.2%; SLMW HA—0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4. This formulation can be made using the process described generally in Example 1.

Example 9: Aloe Barbadensis Leaf Juice—82.9%; Allantoin—0.3%; Micro silver—0.2%; Guar gum—2.5%; Zinc oxide—0.2%; Magnesium Oxide—0.2%; SLMW HA—0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4. This formulation can be made using the process described generally in Example 1.

Example 10: Aloe Barbadensis Leaf Juice—67.9%; Allantoin—0.3%; Micro silver—0.2%; Guar gum—2.5%; Manuka honey—15%; Zinc oxide—0.2%; Magnesium Oxide—0.2%; SLMW HA 0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4. This formulation can be made using the process described generally in Example 1.

Example 11: Aloe Barbadensis Leaf Juice—82.9%; Allantoin—0.3%; Micro silver—0.2%; Carboxymethyl cellulose—2.5%; Zinc oxide—0.2%; Magnesium Oxide—0.2%; SLMW HA—0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4. This formulation can be made using the process described generally in Example 1.

Example 12: Aloe Barbadensis Leaf Juice—67.9%; Allantoin—0.3%; Micro silver—0.2%; Carboxymethyl cellulose—2.5%; Manuka honey—15%; Zinc oxide—0.2%; Magnesium Oxide—0.2%; SLMW HA—0.2%; L-Arginine-HCl—6%; niacinamide (Vitamin B3)—1%; Provitamin B5—1.0%; Beta Glucan—0.5%; Glycerol—5%; Citric Acid to adjust pH to between 6.8 and 7.4. This formulation can be made using the process described generally in Example 1.

In all of the above examples, anyone skilled in the art of mixing ingredients and forming gels knows the order in which the different constituents must be added to prepare the respective formulations.

### REFERENCES

1. ThePharmaLetter. 18 Dec. 1997
2. Fang, R C and Galiano, R C. A review of becaplermin gel in the treatment of diabetic neuropathic foot ulcers. Biologics. 2008 March; 2(1): 1-12.
3. Akita, S, Akino, K, and Hirano, A. Basic Fibroblast Growth Factor in Scarless Wound Healing. Advances in Wound Care. 2013; Vol 2: 44-49.
4. Penn, J W, O'Grobbelaar, A, Rolfe, K J. The role of the TGF-β family in wound healing, burns and scarring: a review. Int J Burn Trauma 2012; 2(1): 18-28.
5. Hardwicke J, Schmaljohann D, Boyce D, Thomas D. Epidermal growth factor therapy and wound healing-past, present and future perspectives. Surgeon. 2008; 6(3): 172-7.
6. Deliaert, A E K, Kerckhove, E V, Tuinder, S, Fieuws, S, Sawor, J. H, Meesters-Caberg, M A, and Hulst, R. The effect of triclosan-coated sutures in wound healing. A double blind randomised prospective pilot study. Journal of Plastic Reconstructive & Aesthetic Surgery, 2008; 62(6):771-3.=5A
7. Ewart, J. How to identify and treat allergies to wound therapies. Wound Essentials. 2015, vol 10 (2), 83-85.=5B
8. Pan S, Yuan C, Tagmount A, Rudel R A, Ackerman J M, Yaswen P, Vulpe C D, Leitman D C. 2016. Parabens and human epidermal growth factor receptor ligand cross-talk in breast cancer cells. Environ Health Perspect 124:563-569.=5c
9. Yadav, K C H, Kumar, J R, Basha, S I, Deshmukh, G R, Gujjula, R, and Santhamma, B. Wound healing activity of topical application of aloe vera gel in experimental animal models. Int J Pharma Bio Sci. 2012; 3(2): 63-72.
10. Park, J H, Park, E J, Yi H S. Wound Healing and Anti-inflammatory Effects of Topical Hyaluronic Acid Injection in Surgical-Site Infection Caused by Staphylococcus aureus. Int J Low Extrem Wounds 2017; 16(3): 202-207.
11. Litwiniuk, M, Krejner, A, Grzela, T. Hyaluronic acid in inflammation and tissue regeneration. Wounds. 2016; 28(3): 78-88.
12. Araújo L U, Grabe-Guimarães A, Mosqueira V C, Carneiro C M, Silva-Barcellos N M. 2010. Profile of wound healing process induced by allantoin”. Acta Cir Bras. 2010; 25(5): 460-6.
13. Esfahani, S A, Khoshneviszadeh, M, Namazi, M R, Noorafshan, A, Geramizadeh, B, Nadimi, E, & Razavipour, S T. (2015). Topical Nicotinamide Improves Tissue Regeneration in Excisional Full-Thickness Skin Wounds: A Stereological and Pathological Study. Trauma monthly. 2015; 20(4): e18193.
14. Wiederholt T, Heise, R, Skazik, C, Marquardt, Y, Joussen, S, Erdmann K, Schroder H, Merk H F, Baron, J M. Calcium pantothenate modulates gene expression in proliferating human dermal fibroblasts. Exp Dermatol 2009; 18 (11): 969-78.
15. Stechmiller, J K, Childress B, Cowan, L. Arginine supplementation and wound healing. Nutr Clin Pract. 2005; 20(1): 52-61.
16. Kirk S J, Hurson M, Regan M C, Holt D R, Wasserkrug H L, Barbul A. Arginine stimulates wound healing and immune function in elderly human beings. Surgery. 1993; 114(2):155-9; discussion 160.
17. Hurson M, Regan M C, Kirk S J, Wasserkrug H L, Barbul A. Metabolic effects of arginine in a healthy elderly population. J Parenter Enteral Nutr. 1995; 19(3): 227-30.
18. Witte M & Barbul A, Arginine Physiology And Its Implication For Wound Healing, Wound Repair & Regeneration 2003 November; 11(6); 419-423.
19. Zandifar, A, Seifabadi, S, Zandifar, E, Beheshti, S S, Aslani, A, Javanmard, S H. Comparison of the effect of topical versus systemic L-arginine on wound healing in acute incisional diabetic rat model. J Res Med Sci 2015; 20(3): 233-238.
20. Kogan S, Sood A, Granick M. Zinc and wound healing: a review of zinc physiology and clinical applications. Wounds. 2017; 29(4): 102-106.
21. Agren, M S. Studies on zinc in wound healing. Acta Derm Venereol Suppl (Stockh) 1990; 154: 1-36.
22. Moore, J. Can zinc oxide have an impact on wound healing? Podiatary Today. 2003; 16(9); 22-25.
23. Lin, P-H, Sermersheim, M, Li, H, Lee, P H U, Steinberg, S M, Ma, J. Zinc in Wound Healing Modulation. Nutrients. 2018; 10: 1-20.
24. Razzaghi, R, Pidar, F, Momen-Heravi, M, Bahmani, F, Akbari, H, Asemi, Z. Magnesium Supplementation and the Effects on Wound Healing and Metabolic Status in Patients with Diabetic Foot Ulcer: a Randomized, Double-Blind, Placebo-Controlled Trial. Biol Trace Elem Res. 2018; 181 (2): 207-215.
25. Majtan, J, Jesenak, M. β-Glucans: multi-functional modulator of wound healing. Molecules. 2018; 23(4): 806.
26. King, B, Barrett, S, K. F. Cutting, K F., Clinical evaluation of a bioactive beta-glucan gel in the treatment of ‘hard-to-heal’ wounds. J Wound Care. 2017; 26(2): 58-63.
27. Percival, S L, and McCarty, S M. Silver and alginates: role in wound healing and biofilm control. Adv Wound Care (New Rochelle), 2015; 4(7): 407-414.
28. Munteanu, A, Florescu, I P, and Nitescu. A modern method of treatment: the role of silver dressings in promoting healing and preventing pathological scarring in patients with burn wounds. J Med Life. 2016; 9(3): 306-315.
29. Yaghoobi, R, Afshin Kazerouni, A, and Kazerouni, O. Evidence for clinical use of honey in wound healing as an anti-bacterial, anti-inflammatory anti-oxidant and anti-viral agent: a review. Jundishapur Journal of Natural Pharmaceutical Products. 2013; 8(3): 100-4.
30. Molan, P, and Rhodes, T. Honey: a biologic wound dressing. Wounds. 2015; 27(6): 141-151.
31. White, R J. Manuka honey in wound management: greater than the sum of its parts? J Wound Care. 2016; 25(9): 1-4.
32. Rath M, Müller I, Kropf P, Closs E I, Munder M. Metabolism via Arginase or Nitric Oxide Synthase: Two Competing Arginine Pathways in Macrophages. Front Immunol. 2014; 5:532.
33. Efron, D T, Most, D, Shi, H P, Tantry, U S, Barbul, A. A Novel Method of Studying Wound Healing. 2001; 98(1); 16-20.
34. Schaffer, M R, U Tantry, Gross, S S, Wasserkburg, H L, Barbul, A. Nitric oxide regulates wound healing. J. Surgical Res. 1996; 63(1); 237 40.
35. Lee, R H, Efron, D, Tantry, U, and Brabul, A. Nitric oxide in the healing wound: a time-course study. J Surg Res. 2001; 101(1): 104-8.
36. Albina, J E, Mills, C D, Henry, W L, Caldwell, M D. Temporal expression of different pathways of L-arginine metabolism in healing wounds. J Immunol. 1990; 144(10): 3877-80.
37. Micro silver BG Product Catalog, Formulation Aid, page 13; https://www.in-cosmetics.com/_novadocuments/2514.

1. A wound healing composition comprising of: Water in an amount of about 60-90%; micro silver in an amount of about 0.1% to about 5%, L-arginine HCl in an amount of about 1.0% to about 20%, glycerol in an amount of 1% to 10%, a gum in an amount of about 0.5% to about 5.0%, all by weight, in a hydrogel.

2. The composition of claim 1, further comprising Manuka honey in an amount of about 1% to about 50%.

3. A wound healing composition comprising of: Aloe barbadensis leaf juice 10% to-90%; micro silver in an amount of about 0.1% to about 5%, L-arginine HCl in an amount of about 1.0% to about 20%, glycerol in an amount of 1% to 10%, a gum in an amount of about 0.5% to about 5.0% all by weight, in a hydrogel.

4. The composition of claim 3, further comprising Manuka honey in an amount of about 1% to about 50%.

5. A wound healing gel composition consisting essentially of: aloe barbadensis leaf juice in an amount of about 10% to about 90%, allantoin in an amount of about 0.01% to about 1.0%, micro silver in an amount of about 0.1% to about 5%, L-arginine HCl in an amount of about 1.0% to about 20%, and at least one of Manuka honey of least 20 UFM+ in an amount of about 1% to about 50%, or locust bean gum in an amount of about 0.5% to about 5.0%, all by weight, in a hydrogel.

6. The composition of claim 5, including both said Manuka honey and said locust bean gum.

7. The composition of claim 6, further comprising: niacinamide (vitamin B3) present in an amount of about 1% to about 15% by weight.

8. The composition of claim 6, further comprising: provitamin B5 present in an amount of about 1% to about 15% by weight.

9. The composition of claim 6, further comprising: at least one zinc oxide or salt present in an amount of about 0.1 to about 20% by weight.

10. The composition of claim 6, further comprising: at least one magnesium oxide or salt present in an amount of about 0.1 to about 20% by weight.

11. The composition of claim 6, further comprising: hyaluronic acid present in an amount of about 0.01 to about 3% by weight.

12. The composition of claim 11, wherein the hyaluronic acid is super low molecular weight hyaluronic acid.

13. A topical wound healing composition comprising: aloe barbadensis leaf juice in an amount of about 50% to about 80%, allantoin in an amount of about 0.1% to about 0.5%, micro silver in an amount of about 0.2% to about 1%, L-arginine HCl in an amount of about 5.0% to about 10%, and either Manuka honey of least UMF20+ in an amount of about 5% to about 15%, or locust bean gum in an amount of about 2.0% to about 5.0%, or both, up to about 10% of a gel former, up to 5% of a thickener, up to about 10% of solvent, up to about 20.0% of an antioxidant, all by weight,

14. The composition of claim 13, wherein the gel former is carboxymethyl cellulose, the thickener is xanthan gum or bentonite clay, the solvent is glycerol and the antioxidant is ascorbic acid, citric acid or tocopherol.

15. The composition of claim 13, further comprising: hyaluronic acid present in an amount of about 0.1 to about 3% by weight.

16. The composition of claim 15, wherein the hyaluronic acid is super low molecular weight hyaluronic acid.

17. The composition of claim 13, further comprising: zinc oxide or a salt thereof present in an amount of about 0.1 to about 20% by weight.

18. The composition of claim 13, further comprising: magnesium oxide or a salt thereof present in an amount of about 0.1 to about 20% by weight.

19. The composition of claim 15, further comprising: niacinamide (vitamin B3) present in an amount of about 1% to about 15%, provitamin B5 present in an amount of about 1% to about 15%, zinc oxide or a salt thereof present in an amount of about 0.1% to about 20%, and magnesium oxide or a salt thereof present in an amount of about 0.1% to about 20%, all by weight.

20. A kit comprising: a topical wound healing composition comprising: aloe barbadensis leaf juice in an amount of about 50% to about 80%, allantoin in an amount of about 0.1% to about 0.5%, micro silver in an amount of about 0.2% to about 1%, L-arginine HCl in an amount of about 5.0% to about 10%, and either Manuka honey of least UMF20+ in an amount of about 5% to about 20%, or locust bean gum in an amount of about 2.0% to about 5.0%, or both, up to about 10% of a cellulose, up to 5% of a thickener, up to about 10% of solvent, up to about 20.0% of an antioxidant, all by weight, and an oral daily dose comprising at least one of L-arginine in an amount of about 1-30 g, Turmeric in an amount of about 1-10 g and piperine in an amount of about 1-50 mg.

21. The kit of claim 20, wherein the topical composition further comprises: hyaluronic acid present in an amount of about 0.1% to about 3%, niacinamide (vitamin B3) present in an amount of about 1% to about 15%, provitamin B5 present in an amount of about 1% to about 15%, at least one of zinc oxide or a salt thereof or magnesium oxide or a salt thereof present in an amount of about 0.1% to about 20%, all by weight.

22. The kit of claim 20, wherein the oral daily dose form is one or more tablets or capsules.

23. A method of treating a wound comprising the steps of applying to a wound of a patient a topical wound healing gel composition comprising: aloe barbadensis leaf juice in an amount of about 50% to about 80%, allantoin in an amount of about 0.1% to about 0.5%, micro silver in an amount of about 0.2% to about 1%, L-arginine HCl in an amount of about 5.0% to about 10%, and either Manuka honey of least UMF20+ in an amount of about 5% to about 15%, or locust bean gum in an amount of up to 10%, or both, up to about 10% of a gel former, up to 5% of a thickener, up to about 10% of solvent, up to about 20% of an antioxidant, all by weight, and orally administering to the patient a daily dose including at least one of L-arginine in an amount of about 1-30 g, Turmeric in an amount of about 1-10 g and piperine in an amount of about 1-50 g, and wherein said topical wound healing gel composition is administered topically once a day and the oral dose is divided into 2-4 daily doses.
